(19) United States
(12) Patent Application Publication
(10) Pub. No.: US 2020/0405613 A1
(43) Pub. Date: Dec. 31, 2020

(54) APPLICATION OF RAT TAIL COLLAGEN IN FIELD OF COSMETICS

(71) Applicant: GUANGDONG ZHONGERJIAN BIOTECHNOLOGY CO., LTD., Guangzhou (CN)

(72) Inventors: Xiaojiang TANG, Guangzhou (CN); Xiaoyu LU, Guangzhou (CN); Wei HU, Guangzhou (CN); Linghu XIAO, Guangzhou (CN)

(73) Assignee: GUANGDONG ZHONGERJIAN BIOTECHNOLOGY CO., LTD., Guangzhou (CN)

(21) Appl. No.: 16/979,539

(22) PCT Filed: May 15, 2019
(86) PCT No.: PCT/CN2019/087013

§ 371 Date: Sep. 10, 2020

(30) Foreign Application Priority Data
Mar. 16, 2018 (CN) 201810218080.6
Dec. 28, 2018 (CN) 201811619038.1

(51) Int. Cl. A61K 8/65; A61Q 19/08; A61Q 19/00; A61Q 19/02

(52) U.S. Cl. CPC A61K 8/65; A61Q 19/08; A61Q 19/02; A61Q 19/007; A61Q 19/008

(57) ABSTRACT

The invention provides a method of applying rat tail collagen in the field of cosmetics. Compared with the conventional collagen on the market currently, in addition to having the characteristics of general collagen, the rat tail collagen still has the advantages of a stable triple helix structure, high collagen activity, low immunogenicity, controllable quality of the source and high biological safety, so radiation sterilization which lowers the biological activity of the collagen may not be needed. As determined from the experiment, the rat tail collagen prepared by the technical solution provided by the present invention has the properties of whitening skin, inhibiting melanin synthesis, resisting oxidation and allergy, repairing hormone skin and removing acne. The method provided by the present invention also overcomes the technical drawbacks that the collagen-contained cosmetics usually have.

### CROSS REFERENCE TO THE RELATED APPLICATIONS

This application is the national stage entry of International Application No. PCT/CN2019/087013, filed on May 15, 2019, which is based upon and claims priority to Chinese Patent Application No. 201811619038.1, filed on Dec. 28, 2018, and Chinese Patent Application No. 201810218080.6, filed on Mar. 16, 2018, and the entire contents of which are incorporated herein by reference.

### TECHNICAL FIELD

The present invention belongs to the field of cosmetic research and development and particularly relates to application of rat tail collagen in the field of cosmetics.

### BACKGROUND

With the high development of economy, modern people, especially modern women, have an increasingly high pursuit for beauty. Where, as having a wide range of sources without adding a chemosynthetic reagent is added, collagen has developed rapidly in the application field of beauty cosmetics and is a novel raw material for high-quality cosmetics and an ideal filler for skin tissues in modern medical cosmetology.

The collagen has developed rapidly in the application field of the beauty cosmetics and can be used as the new raw material for the high-quality cosmetics and the ideal filler for the skin tissues in modern medical cosmetology. The collagen is used in the cosmetics and is favored by the majority of women who love beauty because of the efficacy of nutrition, repairability, moisture retention, compatibility and affinity thereof.

However, in the prior art, collagen used in the field of beauty cosmetics usually comes from an animal that grows in an ordinary environment, microorganisms and parasites carried by the animal cannot be controlled, and the collagen may carry pathogens harmful to a human body and has a safety hazard. A method for killing the collagen pathogens usually requires irradiation, but may cause denaturation of a structure of the collagen so as to lower the biological activity of the collagen. In addition, the collagen derived from fishes or cattles is generally colored and has a big fishy smell, a toner and a flavoring agent are required to be added to affect the experience of a consumer. The commonly used collagen is low in purity self, difficult to purify and complex in purification process, such that the stability of a product is easily lowered, the product is easy to denature and also needs addition of a larger amount of stabilizers and preservatives in storage and application. Therefore, there is an urgent need for safe and effective collagen.

Thus, application of rat tail collagen in the field of cosmetics is developed to overcome the technical defect that the collagen-contained cosmetics usually need the addition of the toner, the flavoring agent, the stabilizer and the preservative, which seriously affect the quality of the cosmetics in the prior art. This becomes an urgent problem for the person skilled in the art to solve.

### SUMMARY

In view of this, the present invention provides application of rat tail collagen in the field of cosmetics to overcome the technical defect that the collagen-contained cosmetics usually need the addition of the toner, the flavoring agent, the stabilizer and the preservative, which seriously affect the quality of the cosmetics in the prior art.

The present invention provides application of the collagen in the field of the cosmetics. The collagen is the rat tail collagen.

Preferably, the rat tail collagen is rat tail collagen and/or mouse tail collagen.

Preferably, a rat and/or a mouse reaches the experimental animal clean grade or the specific pathogen free (SPF) grade.

Preferably, a carrier for application of the rat tail collagen comprises any one or more of a facial mask, an essence, a cream, gel and a spray.

Preferably, the application is application of the rat tail collagen in the field of hormonal facial skin care products and/or desensitizing skin care products.

Preferably, the application is application of the rat tail collagen in the field of acne-removing skin care products.

Preferably, the application is application of the rat tail collagen in the field of anti-wrinkle skin care products.

Preferably, the application is application of the rat tail collagen in the field of moisturizing skin care products and/or lubricating skin care products.

Preferably, the application is application of the rat tail collagen in the field of oil-control skin care products.

Preferably, the application is application of the rat tail collagen in the field of spot-removal skin care products.

Preferably, the application is application of the rat tail collagen in the field of skin repair skin care products.

In the present invention, the rat tail collagen used is collagen extracted from a tail of the rat or the mouse, and an extract stock solution thereof is a colorless and odorless colloidal liquid and is a natural high-purity collagen based on type I collagen. The rat tail collagen is a preferred material cultured with cell adherence and can promote growth of cells. With the feature, the rat tail collagen is also used as a woundplast, an artificial skin and the like for wound healing by people and is a good natural material for skin repair. However, at present, the application of the rat tail collagen in the cosmetics has not been developed.

Due to the small size of the rat or the mouse, the rat or the mouse can be artificially reared under strict control by adopting a barrier housing facility with constant-temperature, constant-humidity and constant-pressure sterilization, microorganisms and parasites of the rat or the mouse can be thoroughly controlled, and the rat or the mouse may not carry pathogens, such as Bovine Spongiform Encephalopathy (BSE), which is harmful to human, irradiation for sterilization is not needed to lower the activity of the collagen, and thus the safety and the effectiveness of the collagen may be ensured. At present, there are clean-grade and SPF-grade experimental rat and mouse supplies, and the conditions for industrial production are available. In particular, the rat tail is rich in collagen content, high in purity and simple in extraction and separation processes; and non-denaturation of the collagen can be ensured with a low-temperature process, and an obtained product is high in purity, less in impurities and low in allergenicity. The study also finds that the rat tail collagen is taken as a main functional raw material of the cosmetics, has the characteristics of safety, effectiveness and stability and can avoid unnecessary addition of the toner, the flavoring agent and the like, and thus the quality of cosmetics is improved.

In summary, the present invention provides the application of the rat tail collagen in the field of the cosmetics. Compared with the conventional collagen on the market currently, in addition to having the characteristics of general collagen, the rat tail collagen still has the advantages of a stable triple helix structure, high collagen activity and low immunogenicity, is controllable in quality of the source and high in biological safety, and radiation sterilization may not be needed to lower the biological activity; and as determined from the experiment, the rat tail collagen prepared by the technical solution provided by the present invention has the properties of whitening skin, inhibiting melanin synthesis, resisting oxidation and allergy, repairing hormone skin and removing acne. The application of the rat tail collagen in the field of the cosmetics provided by the present invention overcomes the technical defect that the collagen-contained cosmetics usually need to add the toner, the flavoring agent, the stabilizer and the preservative to seriously affect the quality of the cosmetics in the prior art.

### DETAILED DESCRIPTION OF THE EMBODIMENTS

The example of the present invention provides application of rat tail collagen in the field of cosmetics and aims at overcoming the technical defect that collagen-contained cosmetics usually need to add a toner, a flavoring agent, a stabilizer and a preservative to seriously affect the quality of the cosmetics in the prior art.

The technical solution in examples of the present invention will be described clearly and completely below. Obviously, the described examples are only a part of the examples of the present invention, rather than all examples. Based on the examples of the present invention, all other examples obtained by the person skilled in the art without creative work shall fall within the protection scope of the present invention.

In order to explain the present invention in more details, the application of the rat tail collagen provided by the present invention in the field of the cosmetics will be specifically described below in conjunction with examples.

### Example 1

This example is a specific example of extracting rat tail collagen from a rat.

A method for extracting the rat tail collagen comprises the following steps:

(1) Taking an SPF-grade rat, cutting off a tail of the rat immediately after euthanizing the rat, putting the tail on an ice cube, peeling off skin, squashing the peeled tail, removing bones and muscles, and placing the tail in 0.01-0.2 mol/L of a Tris-HCl buffered salt solution at the temperature of 0-10° C., wherein the extracted tail collagen is in a shape of a white filament.

(2) Pretreating the above-mentioned filaments for 4-36 h at the temperature of 0-10° C., performing stirring, removing impure proteins, performing draining, and adding 0.05-2 mol/L acetate solution; adding pepsin with a ratio of (1 to 1000)-(1 to 50000), adding in three times, and completing addition in 12-36 h; and continuously performing stirring during the addition until all tail tendons are enzymatically hydrolyzed and become a latex, wherein enzymatic hydrolysis is maintained for 36-144 h.

(3) Centrifuging the latex solution for 10-40 mins at the temperature of 0-10° C. to remove a solidified material that does not have latex like appearance; adding 0.01-0.1 mol/L of an acetate solution in the latex solution by a volume ratio of (1 to 0.5)-(1 to 2); and performing stirring until the ingredients of the latex solution are mixed uniformly.

(4) Adding 0.1-5 mol/L of a NaCl solution in the latex solution, and performing continuous stirring until white flocculent precipitates do not precipitate in the solution anymore; centrifuging the solution for 20-50 min at a high speed; collecting the precipitates; adding the latex solution in 0.1-5 mol/L of the NaCl solution by a volume ratio of 1 to (0.5-1) to 4, and placing the solution for 6-48 h at the temperature of 0-10° C.; and repeating Step (6) the next day.

(5) Collecting the precipitate collected in the Step (6); adding 0.1-5 mol/L of the NaCl solution to precipitate the collagen again after repeated dissolution with 0.01-2 mol/L of an acetate solution. centrifuging the solution for 20-50 mins at a high speed; collecting the precipitates; repeating the step by 1-5 times until the precipitated collagen can be thoroughly dissolved.

(6) Perfusing the thoroughly purified collagen into a dialysis bag with a molecular weight cut-off being ten thousands to perform dialysis for 48-96 h. changing water by 2-8 times every day; dialysing an external liquid with double distilled water; obtaining a collagen stock solution which is colorless and odorless gel with a pH being 4.1-6.5 and a content being controlled to be 1-10 mg/ml; identifying the rat tail collagen as follows: (1) identifying with SDS-PAGE electrophoresis (SDS-polyacrylamide gel electrophoresis), wherein the prepared rat tail collagen takes a rat tail collagen product of the US SIGMA company as a control, and spectrums of the two have no difference, and molecular weights of three bands are 220 KD, 105 KD, and 95 KD respectively; and showing by an analysis on an amino acid content that the rat tail collagen contains: \textcircled{1} amino acid hydroxyproline with collagen characteristics with the content being about 8%; \textcircled{2} the rat tail collagen does not contain two amino acids of cystine and tryptophan, which is consistent with the own characteristics thereof; and \textcircled{3} the content of glycine is close to one third of the total amino acid content, which is consistent with the own characteristics thereof, and thus the prepared rat tail collagen is high in quality and good in purity.

(7) Loading the thoroughly dialyzed collagen solution into small glass bottles of 20 mL, and performing freeze drying on the solution for 48-144 h at the temperature ranging from −40° C.-0° C., wherein a freeze-dried product is a rat tail collagen freeze-dried gauze swab.

### Example 2

This example is a specific example of extracting rat tail collagen from a mouse.

A method for extracting the rat tail collagen comprises the following steps:

(1) Taking a SPF-grade mouse, cutting off a tail of the mouse immediately after euthanasia, putting the tail on an ice cube, peeling off skin, squashing the peeled tail, removing bones and muscles, and placing the tail at 0.01-0.2 mol/L of a Tris-HCl buffered salt solution at the temperature of 0-10° C., wherein the extracted tail collagen is in a shape of white filaments;

(2) Pretreating the above-mentioned filaments for 4-36 h at the temperature of 0-10° C., performing stirring, removing impure proteins, performing draining, and adding 0.05-2 mol/L acetate solution; adding pepsin with a ratio of (1 to 1000)-(1 to 50000), adding in three times, and completing addition in 12-36 h; and continuously performing stirring during the addition until all tail tendons are enzymatically hydrolyzed and become a latex, wherein enzymatic hydrolysis is maintained for 36-144 h.

(3) Centrifuging the latex solution for 10-40 mins at the temperature of 0-10° C. to remove a solidified material that does not have latex like appearance; adding 0.01-0.1 mol/L of an acetate solution in the latex solution by a volume ratio of (1 to 0.5)-(1 to 2); and performing stirring until the ingredients of the latex solution are mixed uniformly.

(4) Adding 0.1-5 mol/L of a NaCl solution in the latex solution, and performing continuous stirring until white flocculent precipitates do not precipitate in the solution anymore; centrifuging the solution for 20-50 mins at a high speed; collecting the precipitates; adding the latex solution in 0.1-5 mol/L of the NaCl solution by a volume ratio of 1 to (0.5-1) to 4, and placing the solution for 6-48 h at the temperature of 0-10° C.; and repeating Step (6) the next day.

(5) Collecting the precipitate collected in the Step (6); adding 0.1-5 mol/L of the NaCl solution to precipitate the collagen again after repeated dissolution with 0.01-2 mol/L of an acetate solution. centrifuging the solution for 20-50 mins at a high speed; collecting the precipitates; repeating the step by 1-5 times until the precipitated collagen can be thoroughly dissolved.

(6) Perfusing the thoroughly purified collagen into a dialysis bag with a molecular weight cut-off being ten thousands to perform dialysis for 48-96 h. changing water by 2-8 times every day; dialysing an external liquid with double distilled water; obtaining a collagen stock solution which is colorless and odorless gel with a pH being 4.1-6.5 and a content being controlled to be 1-10 mg/ml; wherein an identification method of the rat tail collagen is the same as the Step (6) of Example 1.

(7) Loading the thoroughly dialyzed collagen solution into small glass bottles of 20 mL, and performing freeze drying on the solution for 48-144 h at the temperature ranging from −40° C.-0° C., wherein a freeze-dried product is a rat tail collagen freeze-dried gauze swab.

### Example 3

This example is a specific example of preparing a facial mask by using the rat tail collagen stock solution prepared in Example 1.

TABLE 1
Prescription of the facial mask

| Phases | Ingredients | Compositions |
|---|---|---|
| A | Rat tail collagen stock solution | 0.05% |
| B | Sodium hyaluronate | 3% |
| | Coenzyme Q10 | 3% |
| | Serum albumin | 1% |
| | 1,3-butanediol | 5% |
| C | castor oil | 0.5% |
| | Glycerin | 2% |
| D | Water | Margin |

A preparation method is as follows:

(1) adding an appropriate amount of water to dissolve the rat tail collagen in phase A thoroughly for still standing;

(2) dissolving the sodium hyaluronate, the coenzyme Q10, the serum albumin and the 1,3-butanediol in phase B into an appropriate amount of water, heating a solution to dissolve the solution thoroughly, and waiting until the solution is cooled for still standing;

(3) under the ice bath condition, adding a product in the Step (1) dropwise in the solution in the Step (2), and performing stirring for half an hour for fully combination of the product and the solution;

(4) adding the castor oil and the glycerin in phase C in an appropriate amount of water to dissolve;

(5) keeping an ice bath at a low temperature, adding the solution in the Step (3) in a product obtained in the Step (4) dropwise, and quickly stirring the solution for dispersion;

(6) adding all the remaining water in phase D, and performing stirring and vacuumizing to obtain an active stock solution; and

(7) injecting the above-mentioned active stock solution into the surface where the facial mask is placed, and performing sealing forming to obtain a peel-off mask, that is a rat-derived collagen beauty mask.

Compared with common facial masks on the market, the product has two advantages. Firstly, the phase A of the product is a core active ingredient, the phase B is an auxiliary ingredient for exerting the vitality, the two phases have the synergistic effect, the sodium hyaluronate, the coenzyme Q10 and the serum albumin have the effects of lubricating, moisturizing and promoting skin growth, and after the two phases and the rat tail collagen are prepared into a compound formula, a better effect can be obtained. Secondly, the product is stable in process and easy to shape and does not require low humidity refrigeration. In the process, the castor oil, the glycerin and other excipients are controlled to use, so that the obtained facial mask product is uniform in compositions and stable in quality.

### Example 4

This example is a specific example for verifying the effect of the rat tail collagen prepared in Example 2 in inhibiting cell melanin synthesis.

A mouse B16 melanoma cell model is used as an evaluation system for this product. B16 cells are taken and are digested with 0.25% (weight percentage) of trypsin, a product is prepared into a cell suspension with a density of 5*105/mL with an RPMI-1640 a complete medium, the cell suspension is inoculated into a cell culture plate, and the cell culture plate is cultivated in an incubator with 5% (volume percentage) of CO₂ at a temperature of 37° C. After a cultivation is completed, the culture solution of the active collagen stock solution in Example 1 with mass ratios of 5% and 10% are added respectively, and an equal volume of the complete medium is added in a control group for continuous cultivation for 48 h. Contents of melanin in cells are measured separately.

Please refer to Table 2 for the results obtained.

TABLE 2

| Items | Relative contents of melanin |
|---|---|
| Control group | 98.5 |
| 5% of active collagen culture solution | 64.7 |
| 10% of active collagen culture solution | 42.5 |

Results show that the rat tail active collagen of the present invention has the effect of significantly inhibiting cell melanin synthesis and has the whitening effect.

### Example 5

This example is a specific example for verifying the antioxidant effect of the rat tail collagen prepared in Example 1.

In an in-vitro experiment, pyrogallol and a Cu²⁺—H₂O₂ system are used to determine the scavenging activity of the rat tail active collagen on superoxide anion free radicals and hydroxyl radicals. Please refer to Table 3 for results.

The results show that the above-mentioned rat tail active collagen can significantly scavenge the superoxide anion free radicals and the hydroxyl radicals. And there is a certain dose-effect relationship.

TABLE 3

| | Scavenging rate of | Scavenging rate of |
|---|---|---|
| | DPPH free radicals | hydroxyl radicals |
| | IC50, mg/ml | IC50, mg/ml |
| Blank control | 0.05 | 0.03 |
| 5% of active collagen | 2.6 | 3.1 |
| culture solution | | |
| 10% of active collagen | 4.1 | 5.8 |
| culture solution | | |
| 20% of active collagen | 7.3 | 9.9 |
| culture solution | | |
| 40% of active collagen | 10.8 | 13.1 |
| culture solution | | |

### Example 6

This example is a specific example for verifying the anti-allergy effect of the rat tail collagen prepared in the Example 2.

In an in-vitro experiment, pyrogallol and a Cu²⁺—H₂O₂ system are used to determine the scavenging activity of the rat tail active collagen on superoxide anion free radicals and hydroxyl radicals.

Experimental principle: A body produces antibody IgE to an allergen; when invading the body again, the allergen is combined with the IgE antibody to cause FceRI cross-linking, and signal transduction is triggered. The anti-allergic activity of the present invention is verified by designing a hyaluronic acid activity inhibition experiment.

Experimental steps are as follows: 0.2 ml of a 0.25 m mol/L CaCl₂) solution and 0.8 mL of a hyaluronic acid solution are prepared, the solutions are incubated for 25 min at the temperature of 37° C., 0.6 mL of a rat tail active collagen active stock solution of the present invention is added, and a mixture is incubated for 25 min at the temperature of 37° C.; 0.5 mL of a sodium hyaluronate solution is added, and a mixture is incubated for 30 min at the temperature of 37° C.; 0.2 mL of a 0.2 mol/L NaOH solution and 0.6 mL of an acetylacetone solution are added, heating is performed for 15 min, 1.0 mL of an Ehrlich's reagent is added after cooling for dilution without ethanol, and a product is placed for 20 min to develop color, and an absorbance value is determined. Please referring to Table 4 for results obtained.

TABLE 4

| | Rat tail collagen active stock solution | | | | Control |
|---|---|---|---|---|---|
| Groups | 5% | 10% | 20% | 40% | Blank |
| Inhibition | 20.1 | 34.1 | 49.5 | 54.3 | 8.9. |
| rate | | | | | |

Results show that the rat tail collagen active stock solution of the present invention has a high inhibitory rate on hyaluronic acid, indicating that the rat tail collagen active stock solution has the anti-allergic effect.

### Example 7

This example is a specific example for verifying the treatment effect of the rat tail collagen prepared in Example 1 on a hormonal face.

60 patients suffering from hormonal face dermatitis are selected and are randomly divide into four groups for trying the facial masks prepared in the Example 3 and commercially available facial masks for hormonal face treatment respectively, wherein a use method is as follows: the surface of face skin is covered with the facial mask once a day.

Skin repair results are as follows: experiments show that the Examples 1 to 3 of the present invention have the outstanding repair effect on the hormonal faces, and the outstanding curative effect is exerted on the testers after the facial masks are used for 1-3 months, and the patients can make a full recovery after keeping trial for half a year, and the skin returns to normal without rebound.

Wherein please refer to Table 5 for classification of hormonal face disease grades.

TABLE 5

| Hormonal face disease grade | Identification standard |
|---|---|
| 0 | No erythema, normal skin |
| 1 | Mild erythema |
| 2 | Erythema |
| 3 | Erythema, papules and blisters |
| 4 | Edema and bullae |

For skin of the hormonal face, after the product is used for 1-3 days, the erythema becomes smaller, and inflammation gradually disappears; after the product is used for 2 weeks, the allergy characteristic of the skin is gradually alleviated, and the phenomena of dryness in appearance and peeling are relieved; and after the product is used for 3 months, the skin can be thoroughly desensitized, and the appearance and the function of the skin can be returned to a normal skin level.

According to the severity of the hormonal face of the patient, the use period and number of the facial mask can be appropriately increased or decreased. For a mild patient, the effect can be exerted within 1 month generally, and the function of normal skin can be restored. For a severe hormonal face, some patients successfully return to the functions of the normal skin after keeping use the facial mask for 6 months.

### Example 8

This example is a specific example for verifying the treatment effect of the rat tail collagen prepared in Example 1 on a hormonal face.

18 patients with hormonal faces are selected to observe and track the curative effects of the product. The significant skin care effect is exerted without rebound, and the curative effect is significant.

Case 1.

Wang X, female, 26 years old, a nurse. The main symptoms were as follows: a face had a dry skin with black spots being recurred, she obtained medical attention from many places, but the symptoms still were not improved. There were many black spots on the skin surface, and preliminary diagnosis showed that she had chloasma and butterfly spots. A facial mask prepared in the Example 3 was used for external application on the face twice a day.

After the facial mask is used for ten days, dark-brown spots on the face became lighter, and the skin became rosy; and after the facial mask was continuously used for 15 days, the dark-brown spots were no longer obvious, and the skin appeared delicate and shiny, and no recurrence was found during the follow-up.

Case 2.

Zhang XX, female, 29 years old, and a ticket salesperson. The main symptoms were as follows: she had facial erythema and inflammation for many years, no significant effect is obtained after many treatments; it can be seen that there were many papules and erythema on the face, and indurated abscesses are formed; and preliminary diagnosis showed that she had hormonal face acnes. The facial mask prepared in the Example 3 is used for external application on the face three times a day.

After the facial mask is used for ten days, the papules on the face were reduced by 70%, red and swollen calluses were reduced; and after the facial mask was continuously used for 10 days, most of the acnes were subsided, and the skin appeared rosy, the skin appeared delicate and shiny, and no recurrence has been found during the follow-up.

### Example 9

This example is a specific example for verifying the oil control effect of the rat tail collagen prepared in the Example 1.

Experiment principle: an oil level is determined according to the principle of a photometer. A matting tape is used to absorb oil on human skin and undergoes a physical change under the action of the oil, resulting in a change in light transmittance of the tape. Then the photometer is used to measure the quantity of light transmission, and a difference level of skin oil can be measured.

A test instrument: a CuotometerMPA580 oil tester.

A test method is as follows: 20 patients with oily skin faces are selected and are divided into five groups which are a low-dose group, a medium-dose group, a high-dose group, a blank group, and a commercially available oil control facial mask group respectively. Four patients in each group use the facial masks once a day, and the oiliness conditions of the skin of the patients in each group was observed after the facial masks are continuously used for two weeks and four weeks. According to test results of the grease tester, numerical values of measured oil contents are counted, and an average value method is used to analyze the oil control effect of the patients in each group.

Please refer to Table 6 for experimental results obtained.

TABLE 6

| | Two weeks | Four weeks |
|---|---|---|
| 1% of rat tail collagen | 56.4 ± 4.7 | 52.8 ± 5.0 |
| 5% of rat tail collagen | 48.1 ± 5.2 | 40.6 ± 5.1 |
| 10% of rat tail collagen | 37.3 ± 3.9 | 32.1 ± 4.2 |
| Blank group | 80.1 ± 6.2 | 83.1 ± 5.6 |
| Commercially available oil | 52.1 ± 4.1 | 46 ± 5.1 |
| control facial mask | | |

It can be seen from the experimental results that the oil control levels of the medium-dose group and the high-dose group of this product are higher than those of a commercially available product and have significant differences from the blank and positive control groups, indicating that this product has better oil control activity.

It can be concluded from the above technical solution that the technical solution provided by examples of the present invention has the following advantages:

1. The rat tail collagen of the present invention has better safety.

In the technical solution provided by the present invention, the selected rat tails are all derived from rats or mice as clean-grade or SPF-grade experimental animals, so that the quality of animal source is controllable, the rat tails do not have infectious diseases, zoonotic diseases and the like self, the biological safety of the source is are high, and sterilization with irradiation cannot be used to lower the biological activity of the rat tails. Therefore, the obtained rat tail collagen has better biological safety as well as is very low in immunogenicity and in probability of allergic reaction with the skin. In addition, since a production process of the rat tail collagen requires full low temperature control, an original natural triple helix structure cannot be broken; and the obtained collagen is high in purity, stable in structure and difficult to degrade to produce other impurities, such that the biological safety in the application process is further ensured.

2. The activity of the rat tail collagen of the present invention is better.

The rat tail collagen in the present invention has the activities (such as nutrition and moisturizing repairability) of general collagen. In addition, the rat tail collagen has a use as a cell culture medium, and application of the rat tail collagen in promoting cell growth and wound healing and repairing skin has been reported in literature. Therefore, the rat tail collagen has better effects on the hormonal face, acne removal, wrinkle removal, moisture retention, desensitization, spot removal, skin repair and skin lubrication.

In addition, since the rat tail collagen of the present invention has high purity without structural damages, compared with the general collagen in the market, the dosage is lower, and the effect is better.

3. The rat tail collagen has the desensitization effect.

After the rat tail collagen is applied to the skin, a protective film can be formed, the film has relatively high air permeability self and can block irritation of allergens to the skin at the same time, and the rat tail collagen has significantly desensitization effect on the hormonal face after long-term use. The isolating effect is also effective for acne removal and skin ulcers.

4. The rat tail collagen of the present invention is simple in production process and controllable in quality.

The rat tail collagen of the present invention is a colorless and odorless product, fish skin-derived or bovine-derived collagen on a market generally has a large fishy smell, so that a flavoring agent does not need to be added in the product.

Since the rat tail collagen of the present invention is prepared at a low temperature, the obtained product has a stable structure and is not easily denatured, so that any stabilizer and preservative is not needed to be added during storage and application.

An animal extraction preparation process that has been validated in multiple batches is good in reproducibility and high in quality controllability.

In summary, the present invention provides the application of the rat tail collagen in the field of the cosmetics. Compared with the conventional collagen on the market currently, in addition to having the properties of the general collagen, the rat tail collagen also has the advantages of a stable triple helix structure, high biological safety, high collagen activity and low immunogenicity; and obtained by experimental determination that the rat tail collagen prepared by the technical solution provided by the present invention has the properties of whitening skin, inhibiting melanin synthesis, resisting oxidation and allergy, repairing hormonal skin, controlling oil and removing acnes. The application of the rat tail collagen in the field of the cosmetics provided by the present invention overcomes the technical defect that the collagen-contained cosmetics usually need to add the toner, the flavoring agent, the stabilizer and the preservative to seriously affect the quality of the cosmetics in the prior art.

The above are only the preferred examples of the present invention. It should be pointed out that for the person skilled in the art, without departing from the principle of the present invention, several improvements and modifications can be made, and these improvements and modifications also should be regarded as falling within the protection scope of the present invention.

What is claimed is:

1. A method of applying a collagen in cosmetics, wherein the collagen is rat tail collagen.

2. The method according to claim 1, wherein the rat tail collagen is derived from a rat tail and/or a mouse tail.

3. The method according to claim 1, wherein a carrier for the rat tail collagen comprises any one or more of a facial mask, an essence, a cream, a gel and a spray.

4. The method according to claim 1, wherein is application of the rat tail collagen is used in hormonal facial skin care products and/or desensitizing skin care products.

5. The method according to claim 1, wherein the rat tail collagen is used in acne-removal skin care products.

6. The method according to claim 1, wherein the rat tail collagen is used in anti-wrinkle skin care products.

7. The method according to claim 1, wherein the rat tail collagen is used in moisturizing skin care products and/or lubricating skin care products.

8. The method according to claim 1, wherein the rat tail collagen is used in oil-control skin care products.

9. The method according to claim 1, wherein the rat tail collagen is used in spot-removal skin care products.

10. The method according to claim 1, wherein the rat tail collagen is used in skin repair skin care products.
